(19) United States
(12) Patent Application Publication
(10) Pub. No.: US 2015/0246179 A1
(43) Pub. Date: Sep. 3, 2015

(54) Device and method for drug dosing with administration monitoring, in particular for insulin pen integrated with smart phone apps.

(71) Applicants: Nissim Zur, Givataim (IL); Eli Arad, Tzofit (IL)

(72) Inventors: Nissim Zur, Givataim (IL); Eli Arad, Tzofit (IL)

(21) Appl. No.: 14/634,843

(22) Filed: Mar. 1, 2015

Related U.S. Application Data
(60) Provisional application No. 61/946,812, filed on Mar. 2, 2014.

(51) Int. Cl. A61M 5/315

(52) U.S. Cl. CPC A61M 5/3157; A61M 5/31535; A61M 2205/3368; A61M 2230/201; A61M 2205/52

(57) ABSTRACT

A hand held drug administration unit that may include a controller, sensors, a wireless transceiver, a memory unit, and a mechanical dosage and injection control unit that is controlled by a user; wherein the sensors are configured to generate detection signals indicative of a progress of a drug provision process; wherein the controller is configured to process the detection signals and to determine the progress of the injection process and to provide a notification regarding the progress of the injection process

### RELATED APPLICATION

This application claims priority from U.S. provisional patent Ser. No. 61/946,812 filing date Mar. 2, 2014 which is incorporated herein by reference.

### BACKGROUND OF THE INVENTION

The present invention relates to managing diabetes symptoms, and more particularly, to a device and method for drug dosing with administration monitoring, in particular for insulin pen integrated with smart phone apps for a diabetes patient.

The prior art includes devices for monitoring the use of insulin injection pen to control blood glucose levels of diabetes patients and devices for administering insulin to control blood glucose levels.

The diabetes patient who needs to use the insulin injection pen has to remember very complex instructions prior the use of insulin injection pen according to the insulin injection pen manufacturer.

For, example, injecting Insuline using the Novolin® 30/70 vial of Novo Nordisk A/S involve the following:
1. Check to make sure that you have the correct type of insulin.
2. Look at the vial and the insulin. The insulin should be a cloudy or milky suspension. The tamper-resistant cap should be in place before the first use. If the cap had been removed before your first use of the vial, or if the precipitate (the white deposit at the bottom of the vial) has become lumpy or granular in appearance or has formed a deposit of solid particles on the wall of the vial, do not use it, and call Novo Nordisk at 1-800-727-6500.
3. Wash your hands with soap and water. If you clean your injection site with an alcohol swab, let the injection site dry before you inject. Talk with your healthcare provider about how to rotate injection sites and how to give an injection.
4. If you are using a new vial, pull off the tamper-resistant cap. Wipe the rubber stopper with an alcohol swab.
5. Roll the vial gently 10 times in your hands to mix it. This procedure should be carried out with the vial in a horizontal position. The rolling procedure must be repeated until the suspension appears uniformly white and cloudy. Shaking right before the dose is drawn into the syringe may cause bubbles or froth, which could cause you to draw up the wrong dose of insulin.
6. Pull back the plunger on the syringe until the black tip reaches the marking for the number of units you will inject.
7. Push the needle through the rubber stopper of the vial, and push the plunger all the way in to force air into the vial.
8. Turn the vial and syringe upside down and slowly pull the plunger back to a few units beyond the correct dose.
9. If there are any air bubbles, tap the syringe gently with your finger to raise the air bubbles to the top. Then slowly push the plunger to the marking for your correct dose. This process should move any air bubbles present in the syringe back into the vial.
10. Check to make sure you have the right dose of Novolin 70/30 in the syringe.
11. Pull the syringe with needle out of the vial's rubber stopper.
12. Your doctor should tell you if you need to pinch the skin before inserting the needle. This can vary from patient to patient so it is important to ask your doctor if you did not receive instructions on pinching the skin. Insert the needle into the skin. Press the plunger of the syringe to inject the insulin. When you are finished injecting the insulin, pull the needle out of your skin. You may see a drop of Novolin 70/30 at the needle tip. This is normal and has no effect on the dose you just received. If you see blood after you take the needle out of your skin, press the injection site lightly with a piece of gauze or an alcohol wipe. Do not rub the area.

Known blood glucose monitors take many forms. For example, one type of monitor is implanted in a patient and transmits blood glucose readings, to an external smart phone apps display, more or less continuously. Other devices require the patient to take periodic blood samples for analysis by the glucose monitor. In the latter type of device, the patient typically lances a finger and places a blood sample on a medium such as a test strip. The monitor analyzes the test strip and provides a digital readout of the blood glucose level on a monitor smart phone apps display. Depending on the patient's blood glucose level, it may or may not be necessary to administer a dose of insulin. Insulin delivery devices also take many forms. Broadly speaking, insulin delivery can be either essentially automatic by permanently attaching the patient to an insulin pump, or as-needed by using an injection device (such as a hypodermic needle) with which the patient injects an amount of insulin determined according to a predetermined protocol when the measured blood glucose level is outside an acceptable range.

Many devices and systems seek to automate diabetics blood glucose control protocols by computerizing conventional devices so that insulin dosages can be automatically determined and delivered with minimum intervention by the patient. The following references illustrate some typical examples of such devices and systems:

U.S. Pat. No. 4,731,726, U.S. Pat. No. 5,019,974, U.S. Pat. No. 5,536,249, U.S. Pat. No. 5,593,390, U.S. Pat. No. 5,728,074, U.S. Pat. No. 5,822,715, U.S. Pat. No. 5,840,020, U.S. Pat. No. 5,925,021, U.S. Pat. No. 6,192,891, U.S. Pat. No. 6,544,212 U.S. Pat. No. 6,875,195, U.S. Pat. No. 6,906,802, U.S. Pat. No. 7,427,275, U.S. Pat. No. 7,534,230, U.S. Pat. No. 7,591,801, U.S. Publ. No. 2008/0306434, U.S. Publ. No. 2010/0010330, European. App. No. 1 102 194.

Devices disclosed in U.S. Pat. No. 5,728,074 embodies the “as-needed” type of insulin delivery approach. Some of these disclosed devices could be particularly useful because they provide a variety of functions that a diabetic would undoubtedly find helpful in managing his or her disease. For example, the disclosed embodiments include devices that combine an insulin injection mechanism and a blood glucose monitor, such as the “pen-type injector”. This device has at one end a removable cap that conceals a hypodermic needle for insulin injection and a lancet mechanism used by the patient to prick a finger to obtain a blood sample for analysis by a test strip on the injector housing. U.S. Patent Pub. No. 2010/0010330 exemplifies the type of system that employs a blood glucose sensor implanted in the patient to provide continuous glucose level data to a bedside monitoring system that controls an insulin infusion pump. The system can includes software that determines if the patient's blood glucose level is at a dangerously low level and can alert 911 or other medical emergency response provider. While this feature enhances patient safety, it has a significant drawback in that the patient is tethered to the monitoring system.

Many diabetics lead relatively active lives, and for them being tethered to a monitoring system are obviously not acceptable. These patients require a treatment regimen that enables them to maintain a normal lifestyle by minimizing limitations that might otherwise be imposed by their diabetes. Even though existing devices and systems permit such patients to closely monitor their own blood glucose levels, and thus minimize the risk of becoming hypoglycemic or hyperglycemic at any given time, a diabetes patient still can experience either condition without much warning. Hypoglycemia can be particularly dangerous because it can impair cognitive functions, so a patient with a low blood glucose level can become disoriented and confused very rapidly. If the patient's blood glucose level is not corrected in time, he or she can lapse into a coma and even die before being able to take necessary corrective action. By the same token, hyperglycemia, while less likely than hypoglycemia to present an emergency situation, can nonetheless be dangerous. Accordingly, devices that rely on the patient to take appropriate steps after determining his or her own blood glucose level would have greater utility if they could automatically take action to preempt the potentially serious consequences of rapid changes in blood glucose levels.

### SUMMARY OF THE INVENTION

According to an embodiment of the invention there may be provided a hand held drug administration unit that may include a controller, sensors, a wireless transceiver, a memory unit, and a mechanical dosage and injection control unit that is controlled by a user; wherein the sensors are configured to generate detection signals indicative of a progress of a drug provision process; wherein the controller is configured to process the detection signals and to determine the progress of the injection process and to provide a notification regarding the progress of the injection process.

The controller, the sensors and the wireless transceiver may be integrated within a housing of the hand held drug administration unit or are connected to the housing of the hand held drug administration unit.

The hand held drug administration unit may be a drug injection pen.

The sensors may include an orientation sensor configured to monitor an orientation of the hand held drug administration unit.

The controller may be configured to determine, based upon detection signals from the orientations sensor, whether the user performs predetermined movements that are mandatory to the drug provision process.

The orientation sensor may be a three axis accelerometer.

The sensors may include at least one mechanical dosage and control unit sensor that may be configured to sense a movement of the mechanical dosage and control unit.

The controller may be configured to determine, based upon, at least, detection signals from the at least one mechanical dosage and control unit sensor, a target amount of dosage to be administered during the drug provision process and an actual amount of drug that may be administered by the user.

The at least one mechanical dosage and control unit sensor may include a magnetic element connected to the mechanical dosage and control unit and a static magnetic flux reader.

The sensors may include a temperature sensor for sensing when the drug is injected, wherein the controller may be configured to determine, based upon, at least, detection signals from the temperature sensor and at the least one mechanical dosage and control unit sensor, a target amount of dosage to be administered during the drug provision process and an actual amount of drug that may be administered by the user.

The sensor may include a temperature sensor for sensing when the drug is being injected.

The memory unit stores a set of instructions to be followed by the user when administering the drug, wherein the controller may be configured to determine whether the user follows the instructions and send an alert when the user deviates from the instructions.

The hand held drug administration unit may include a man machine interface and/or may wirelessly communicate with a user device that has a man machine interface for providing to the user indications about the progress of the drug administration process.

The controller may be configured to receive information from a user monitor about a status of the user and to send an alert if the user did not administer the drug in accordance to the status of the user.

The controller may be configured to generate an alert may be the user did not administer a drug when the status of the user requires an administration of the drug.

The controller may be configured to administer an alert when the user sets the mechanical dosage and injection control unit to administer an amount of drug that may be too high to the status of the user.

According to an embodiment of the invention there may be provided a method for administering drug, the method may include generating by sensors of a hand held drug administration unit, detection signals indicative of a progress of a drug provision process; processing, by a controller of the hand held drug administration unit, the detection signals to determine the progress of the injection process and, providing a notification regarding the progress of the injection process. According to an embodiment of the invention there may be provided a kit that may include a controller, sensors, a wireless transceiver, a memory unit. When at least one of the sensors is coupled to a mechanical dosage and injection control unit (that is controlled by a user and belongs to a hand held drug administration unit), the at least one of the sensors is configured to generate detection signals indicative of a progress of a drug provision process; and the controller is configured to process the detection signals and to determine the progress of the injection process and to provide a notification regarding the progress of the injection process.

### BRIEF DESCRIPTION OF THE DRAWINGS

The objects of the invention will be better understood from the detailed description of its preferred embodiments which follows below, when taken in conjunction with the accompanying drawings, in which like numerals and letters refer to like features throughout. The following is a brief identification of the drawing figures used in the accompanying detailed description.

FIG. 1 is a schematic diagram of an insulin injection pen according to an embodiment of the invention;

FIG. 2 illustrates sample insulin injection pen parts, prior to add-on of the monitor device;

FIG. 3 illustrates sample insulin injection pen parts, rear side, with the added of the monitor device according to an embodiment of the invention;

FIG. 4 is a schematic electronic diagram of the insulin injection monitoring device according to an embodiment of the invention;

FIG. 5 is a snapshot of a screen of smart phone that executed an application according to an embodiment of the invention;

FIG. 6 is a snapshot of a screen of smart phone that executed an application according to an embodiment of the invention;

FIG. 7 is a snapshot of a screen of smart phone that executed an application according to an embodiment of the invention;

FIG. 8 is a snapshot of a screen of smart phone that executed an application according to an embodiment of the invention;

FIG. 9 is a snapshot of a screen of smart phone that executed an application according to an embodiment of the invention; and

FIG. 10 illustrates a method according to an embodiment of the invention.

One skilled in the art will readily understand that the drawings are not strictly to scale, but nevertheless will find them sufficient, when taken with the detailed descriptions of preferred embodiments that follow, to make and use the present invention.

### DETAILED DESCRIPTION OF PREFERRED EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method.

Any reference in the specification to a system should be applied mutatis mutandis to a method that may be executed by the system.

There may be provided a wireless micro-small device attached or built inside an insulin injection pen, with sense usage ability of the insulin injection pen. The device includes sensors to detect insulin dosing, pen storage temperature, humidity, accelerometer positing, gyro rotation, human body temperature, wireless link to wireless devices like glucometers and smart phones, pen needle taping for clear air bubble. The device has also the entire pen manufacturer complex administration guide and monitor its action by the user.

The device utilizes its wireless link to smart phone for voice guide, dose setting, internee and care taking reporting and emergency alerting.

The device has a wireless connection for blood glucose monitoring. The device's microprocessor, calculates an insulin dosage appropriate to the detected blood glucose level of a particular user and monitor the insulin injection mechanism to administer the calculated insulin dosage. The device automatically informs a remote emergency service provider if the microprocessor determines that the detected blood glucose level presents a potential danger to the user or if the user did not use the pen or when the user pressed the emergency button.

The device's microprocessor also calculates treatment regimens based on the detected blood glucose level, based on the manufacturer recommendation level and smart phone apps displays the treatment regimens on the smart phone apps display. The device's smart phones apps can use the smart phone GPS receiver detecting the location of the device, for transmitting it to the remote emergency service.

It is an object of the present invention to improve on known techniques involving insulin injection pen self-administration of appropriate therapy to adjust glucose levels after a patient tests his or her own blood glucose level. One important aspect of the invention provides an automatic all action needed to do with the pen before the injection and added value by the wireless channel to user smart phone apps.

In accordance with a first aspect of the invention, a device attached to insulin injection pen or integrated inside, connected to smart phone and to wireless blood glucose monitoring device and insulin injection pen integrated into a single unit for testing and treating diabetes symptoms in a user, comprises a housing of a size suitable for transport in a handbag or clothing pocket of the user. An insulin injection mechanism within the housing for permitting the user to self-administer an insulin injection, a microprocessor within the housing for calculating an insulin dosage appropriate to the wirelessly detected blood glucose level and setting the insulin injection mechanism monitor to administer the calculated insulin dosage, a smart phone apps display for displaying the detected blood glucose level and the calculated insulin dosage, and a communication device within the housing and under the control of the microprocessor for automatically informing a remote emergency service provider if the microprocessor determines that the detected blood glucose level presents a potential danger to the user, or the user pressed the emergency button.

In accordance with more specific embodiments of the invention, such a unit further comprises at least one manual input device operable by the user in conjunction with information on the smart phone apps display for providing a user interface for permitting the user to control predetermined operations of the unit. A particularly advantageous embodiment comprises a GPS receiver within the smart phone for detecting the location of the device, wherein the smart phone transmits information regarding the location of the pen to the remote emergency service.

In accordance with more specific embodiments of the invention, such as the smart phone apps can get guide the user by human voice about the usage on the pen. The insulin injection pen microcontroller has several sensors that detect the pen usage and send that info to the smart phone for proper human voice narration.

An additional aspect of the invention includes a method of monitoring the insulin injection pen following the manufacture complex guide by many sensors inside the device so the user can do self-injection.

In accordance with more specific method aspects of the invention, the storage device stores a first threshold representing a blood wireless glucose level below which the patient is severely hypoglycemic and may be disoriented or comatose, and a second threshold above which the patient is severely hyperglycemic and may require immediate medical intervention, and the method further includes setting a time period by which the patient must provide an input to the monitoring device if the detected blood wireless glucose level is below the first threshold or above the second threshold before automatically activating the communication device. In another variation, the monitoring device further comprises an insulin injection mechanism within the housing for permitting the user to self-administer an insulin injection, and the method further includes determining if the detected wireless blood glucose level indicates that the patient is hypoglycemic or hyperglycemic, and if the patient is hypoglycemic, instructing the patient to ingest an amount of at least one blood glucose producing substance based on the detected blood glucose level, or if the patient is hyperglycemic, calculating an insulin dosage appropriate to the detected blood glucose level and using the insulin injection mechanism to set an amount of insulin to be injected based on the detected blood glucose level.

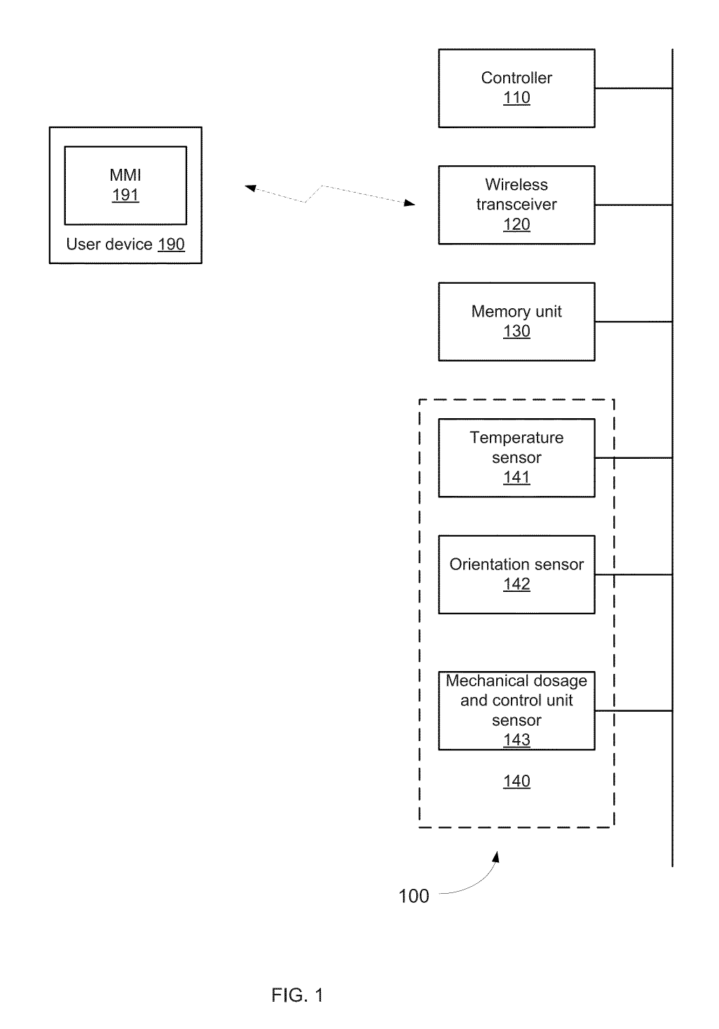

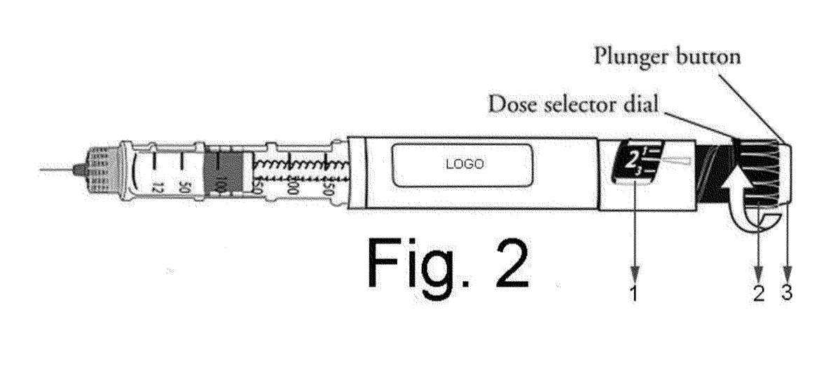

FIGS. 1-2 illustrate a hand held drug administration unit (100 in FIG. 1) that may include a controller 110, sensors (such as temperature sensor 141, orientation sensor 142 and mechanical dosage and control unit sensor 143), a wireless transceiver 120, a memory unit 130, and a mechanical dosage and injection control unit (such as dose selector dial 2 and plunge button 3 of FIG. 2) that is controlled by a user.

In FIG. 2 the mechanical dosage and control unit sensor 143 may include a magnet (denoted 6) connected to the plunge button 3 and (not shown) a magnetic measurement element that may sense the magnetic flux induced by the magnet.

The sensors 140 are configured to generate detection signals indicative of a progress of a drug provision process. The controller 110 is configured to process the detection signals and to determine the progress of the injection process and to provide a notification regarding the progress of the injection process.

The controller, the sensors and the wireless transceiver may be integrated within a housing of the hand held drug administration unit or are connected to the housing of the hand held drug administration unit.

FIG. 1 illustrates a user device 190 (such as but not limited to a mobile phone) that has a man machine interface (MMI 191) that is wirelessly coupled to the wireless transceiver.

Samples of usage instructions by an insulin injection pen manufacturer. Every user must follow this complex preparing the insulting injection pen before self-injection. Other insulin injection pen manufacture has similar instructions.

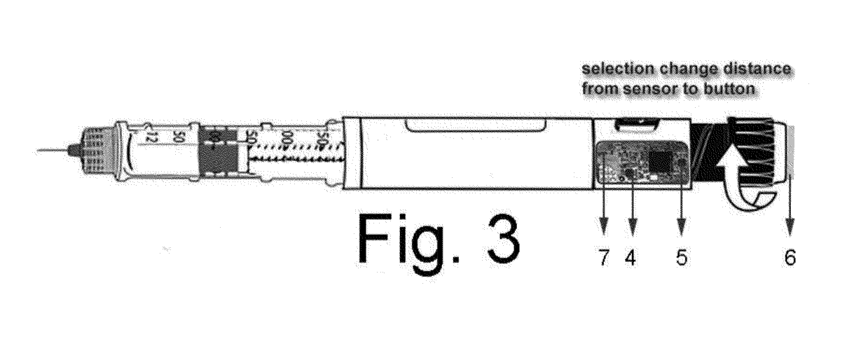

“Let the insulin reach room temperature before you use it (See diagram A). This makes it easier to mix”. The device is a tiny PCB attached to pen (FIG. 3—denoted 4).

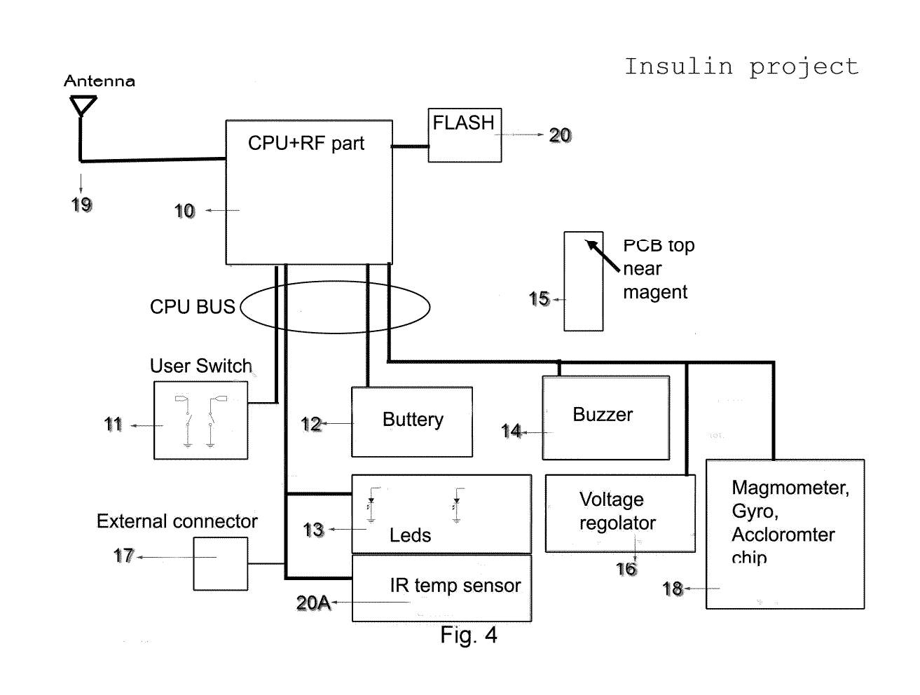

The device has a temperature sensor inside the device's microcontroller (FIG. 4 denoted 10) and inside the device sensor (FIG. 4—denoted 18). The device's microcontroller stores inside the device a flash storage of the temperature. If temperature at any point of time at the past deviate the recommended by the manufacturer, it marks this pen as bad one, and should not be used. And report with error by buzzer tone on all attempts to use it. The smart phone apps will report that accordingly.

“Roll the pen between your palms 10 times it is important that the pen is kept horizontal” (See diagram B).

The device (FIG. 3 denoted 4) with its Gyro, Accelerometer, and Magnetometer sensor (FIG. 3—denoted 5) detects rolling by the Gyro, while keep pen horizontal by the accelerometer. The device microcontroller counts and beeps accordingly.

“Then gently move the pen up and down ten times between position 1 and 2 as shown, so the glass ball moves from one end of the cartridge to the other. Repeat rolling and moving the pen until the liquid appears white and cloudy”. (See diagram C).

The device (FIG. 3—denoted 4) with its Gyro, Accelerometer, and Magnetometer sensor (FIG. 3 denoted 5) detects pen up and down by the accelerometer. The device microcontroller counts and beeps accordingly.

Caution: “Before you inject, there must be at least 12 units of insulin left in the cartridge to make sure the remaining insulin is evenly mixed. If there are less than 12 units left”.

The device (FIG. 3—denoted 4) with its Gyro, Accelerometer, and Magnetometer sensor (FIG. 3—denoted 5) detects the dosage selecting dial by proximity to its sensor Magnetometer since on the pen button locate a magnet (FIG. 3—denoted 6). The device's microcontroller stores all past injection dosage from this pen and calculates the remaining insulin units left in the pen insulin cartridge. If the calculated sum is less than 12 units left, the device will beep error tone accordingly, and the smart phone will play alarm human voice message.

Giving the air shot before each injection, small amounts of air may be collected in the cartridge during normal use. To avoid injecting air and to make sure you take the right dose of insulin “Be sure you take the right dose of insulin:”

The device can detect the amount been taken by the user, by movement sensing of its sensor (FIG. 3—denoted 5).

“Turn the dose selector to select 2 units”. “Mix Pen with the needle pointing up. Tap and Hold your insulin injection pen cartridge gently with your fingers a few times to make any air bubbles collect at the top of the cartridge”, “Keep the needle pointing upwards, press the push-button all the way in”.

The device (FIG. 3—denoted 4) with its Gyro, Accelerometer, and Magnetometer sensor (FIG. 3—denoted 5) detects the dosage selecting dial by proximity to its sensor Magnetometer since on the pen button is located a magnet (FIG. 3—denoted 6). The device's microcontroller translates the Magnetometer detection units to dose selector to select 2 units. The device's microcontroller uses the sensor (figure)—denoted 5) Accelerometer to detect the pen pointing up. The Accelerometer also reports to microcontroller tapping on the pen, by its “tapping” detection. Fail to do so will alert the user by error buzzer beep.

1. “The dose selector returns to 0. A drop of insulin should appear at the needle tip. If not, change the needle and repeat the procedure no more than 6 times. If you do not see a drop of insulin after 6 times, do not use the insulin injection pen”

The device (FIG. 3—denoted 4) with its Gyro, Accelerometer, and Magnetometer sensor (FIG. 3—denoted 5) detect the dosage selecting dial returns to 0 by proximity to its Magnetometer sensor, since on the pen button is located a magnet (FIG. 3 denoted 6). The device's microcontroller translates the Magnetometer detection units to dose selector to select 0 units. The device's microcontroller uses the sensor (FIG. 3 denoted 5) Accelerometer to detect the pen pointing up. The Accelerometer also reports to microcontroller tapping on the pen, by its “tapping” detection. The device's microcontroller counts the user trials, if it counts more than 6 times, it notes this pen as bad one, and should not be used. It reports with error buzzer tone on all attempts to use it. The smart phone apps will report that accordingly.

“Turn the dose selector to the number of units you need to inject. The pointer should line up with your dose”.

“The dose can be corrected either up or down by turning the dose selector in either direction until the correct dose lines up with the pointer”.

When turning the dose selector from 0 to up, the device buzzes (FIG. 4 denoted 14) OK beep when reach the right selection. The device has also 2 leds (FIG. 4—denoted 13), while dial the dosage the “below” led will light.

If pass the right dosage selection, the “above” led will light. So user can easily select the dial when the 2 leds are closed (FIG. 4—denoted 13), and the buzzer beep OK. The device gets the right dosage selection by translating the glucose level been sent from the wireless glucometer or from the smart phone apps.

Giving the Injection:

“Do the injection exactly as shown to you by your healthcare provider. Your healthcare provider should tell you if you need to pinch the skin before injecting. Wipe the skin with an alcohol swab and let the area dry. Insert the needle into your skin. Inject the dose by pressing the push-button all the way in until the 0 lines up with the pointer”. “Be careful only to push the button when injecting. Turning the dose selector will not inject insulin”.

The device (FIG. 3 denoted 4) has an IR temperature sensor (FIG. 4 denoted 20A). The device's microcontroller (FIG. 4—denoted 10) receives over its I2C bus the human body IR radiation level, proportional to the distance to human skin. By calculating the IR data the device's microcontroller with the Magnetometer information detects the injection. The Magnetometer reports the dosage dial from its “far locating” to “zero”.

If the human body temperature is out of the expected range, and an alert message can be send using the smart phone apps. All dosage injection, with all pen storage temperature and human skin temperature, stored in the device's flash and report to the smart phone. The smart phone apps will report that accordingly to the care taker over the internet.

7. “Insert the needle into the skin for at least 6 seconds, and keep the push-button pressed all the way in until the needle has been pulled out from the skin”. “This will make sure that the full dose has been given”.

The device has an IR temperature sensor (FIG. 4—denoted 20A). The device's microcontroller (FIG. 4—denoted 10) receives over its I2C bus the human body IR radiation level, proportional to the distance to human skin. By calculating the IR data the device's microcontroller with the Magnetometer information detects the injection. The Magnetometer reports the dosage dial from its “far locating” to “zero”. The device's microcontroller (FIG. 4 denoted 10) calculates the time needle remain in skin. It beeps 6 seconds to inform the user not to take the needle out. If The device's microcontroller (FIG. 4—denoted 10) detects by the IR temperature sensor (FIG. 4—denoted 20A) the temperate dropped before 6 second, that means that the user pulled out the needle too early and report will be send to smart phone. The smart phone apps will report that accordingly to the care taker over the internet

FIG. 2 Illustrates sample insulin injection pen parts prior to add-on of the monitor device.

The pen dosage selection dial windows (FIG. 2—denoted 1).

The pen dosage selection dial wheel (FIG. 2—denoted 2).

The pen injection button (FIG. 2—denoted 3). The user presses this button to inject himself the dosages insulin he previously selected.

FIG. 3 Illustrates sample insulin injection pen parts, rear side, with the added of 2 parts:

The device PCB (FIG. 3—denoted 4) which its detailed schematics is described at (FIG. 4).

The device's tiny flat magnet (FIG. 3—denoted 6) attached to pen button (FIG. 2—denoted 3).

The device PCB has a sensor with several embedded functions (FIG. 4—denoted 18) name MPU-9250.

The MPU-9250, 3-axis silicon monolithic Hall-effect magnetic sensor with magnetic concentrator. This sensor has the ability to sense the distance between the sensor location to magnet located on the flat magnet (FIG. 3—denoted 6) attached to pen button (FIG. 2—denoted 3). When the user dials selection the flat magnet (FIG. 3—denoted 6) moves away from the sensor. Therefore a lower magnetic field is sensed by MPU-9250, and the value is reported to the PCB microcontroller.

The device PCB has also IR sensor (FIG. 3—denoted 7 FIG. 4 20A). This IR sensor pointing to the pen needle. This IR temperature sensor measures the temperature of the user skin without the need to make contact with the user skin. This sensor uses a thermocouple to absorb measured and uses the corresponding change in thermocouple voltage to determine the user skin temperature. When the user moves his hand to inject the insulin the IR sensor (FIG. 4—denoted 20A) reports over I2C bus to the PCB microcontroller (FIG. 4—denoted 10) rising of temperature. The peak value is when the needle is inside the user body. And lower temperature reported when the needle is taken out. This effect of temperature higher and lower is normal since the user skin radiates a fix level of IR, and when IR sensor is away, it detects lower IR radiation. The PCB Microcontroller temperature reports to detect the 6 second the needle must be remaining inside the user skin. The Microcontroller sound beep every second laps to notify the user. The Microcontroller also monitors the magnetic sensor during this 6 second to detect that the user is actually pressing the button. If the Microcontroller detects by the magnetic sensor that the pen button did not pressed all the way in (to dial value 0), it will keep beeping so the user will continue pressing the pen button.

FIG. 4 is a schematic illustration of the insulin injection monitoring device. One skilled in the art will readily understand that the drawings. All the components are easily retrieve from the manufacturer web site using the internet.

Element 10 of FIG. 4 is a microcontroller device by the name—CSR1012 CSR μEnergy enables ultra-low-power connectivity and basic data transfer for applications previously limited by the power consumption, size constraints and complexity of other wireless standards. CSR1012 QFN provides everything required to create a Bluetooth low energy product with RF, baseband, MCU, qualified Bluetooth v4.x specification stack and customer application running on a single IC. building an ecosystem using Bluetooth low energy. CSR is the industry leader for Bluetooth low energy, also known as Bluetooth Smart. Bluetooth Smart enables connectivity and data transfer to leading smartphone, tablet and personal computing devices including Apple iPhone, iPad, iPod and Mac products and leading Android devices.

The device's microcontroller has the ability to sleep in order to get low power consumption and wake up when one of the sensors send wake up interrupt. The IR (FIG. 4—denoted 20A) sensor or the MPU-9250 sensor (FIG. 4—denoted 18). The Microcontroller can also wake up by internal timer and on getting a wireless transmitting from the smart phone or a wireless glucometer.

Element 11 of FIG. 4 is the device's buttons. Sw1 is the user wake up button. On the very first use of the device, when the pen is just out of its box, the user presses this SW1 switch to wake up the system to detect the user smart phone and the wireless glucometer. Sw2 is an emergency alert button and the user can press on it to make his smart phone calling for help. Sw1 is also used as function selection when in the devices.

Element 12 of FIG. 4 is the contact to micro small battery. In this case a CR927 coin size battery is in use.

Element 13 of FIG. 4 is the device's led1 and led2. Led1 and led2 are used for guiding the user to dial the right dosage selection. The device receives automatically wirelessly the last glucose level from wireless glucometer or from the user smart phone. When the dosage level is below the needed selection led 1 light, when the dosage is above the needed dosage selection level led2 light. When it is on the selection, both leds blink rapidly and then close.

Element 14 of FIG. 4 is the device's buzzer. The microcontroller is driving the buzzer directly and able to sound many different sounds, and OK sound, alert sound. Over dosage selection sound, end of injection sound, receiving wirelessly new glucose level etc.

Element 15 of FIG. 4 is the device's components placement. It is one of the options to place the components on the PCB.

Some important aspects of the parts placement are: The proximity of the MPU-9250 sensor (FIG. 4—denoted—element 18) near the button magnet (FIG. 3—denoted 6); The proximity of the IR sensor near the pen needle; a clear PCB area for the antenna; a simple way to change the device's battery.

Element 16 of FIG. 4 is an option to add external power supply to the device. In some cases it is needed to be powered from external source and not from the inner battery. During development or when the device's PCB is in used inside other device. For example an automatically insulin pump device that can be controlled from external source as smart phone. The insulin pump will provide the need power to (FIG. 4—denoted 16).

Element 17 of FIG. 4 is an option to add a connector to extend the device's functionality and bridge it to other devices as automatically insulin pump devices.

Element 18 of FIG. 4 is the device's MPU-9250 sensor features are:

Gyroscope Features (Non-Limiting Example)
The triple-axis MEMS gyroscope in the MPU-9250 includes a wide range of features:
   Digital-output X-, Y-, and Z-Axis angular rate sensors (gyroscopes) with a user-programmable e full-scale range of ±250, ±500, ±1000, and ±2000°/sec and integrated 16-bit ADCs
   Digitally-programmable low-pass filter
   Gyroscope operating current: 3.2 mA
   Sleep mode current: 8 μA
   Factory calibrated sensitivity scale factor
   Self-test

Accelerometer Features (Non-Limiting Example)
The triple-axis MEMS accelerometer in MPU-9250 includes a wide range of features:
   Digital-output triple-axis accelerometer with a programmable full scale range of ±2 g, ±4 g, ±8 g and
±16 g and integrated 16-bit ADCs
   Accelerometer normal operating current: 450 μA
   Low power accelerometer mode current: 8.4 μA at 0.98 Hz, 19.8 μA at 31.25 Hz
   Sleep mode current: 8 μA
   User-programmable interrupts
   Wake-on-motion interrupt for low power operation of applications processor
   Self-test

Magnetometer Features (Non-Limiting Example)
The triple-axis MEMS magnetometer in MPU-9250 includes a wide range of features:
   3-axis silicon monolithic Hall-effect magnetic sensor with magnetic concentrator
   Wide dynamic measurement range and high resolution with lower current consumption.
   Output data resolution of 14 bit (0.6 μT/LSB) or 16 bit (15 μT/LSB)
   Full scale measurement range is ±4800 μT
   Magnetometer normal operating current: 280 μA at 8 Hz repetition rate
   Self-test function with internal magnetic source to confirm magnetic sensor operation on end

Additional Features (Non-Limiting Example)
The MPU-9250 includes the following additional features:
Auxiliary master I2C bus for reading data from external sensors (e.g. glucometer sensor)
3.5 mA operating current when all 9 motion sensing axes and the DMP are enabled
VDD supply voltage range of 2.4-3.6V
VDDIO reference voltage for auxiliary I2C devices
Smallest and thinnest QFN package for portable devices: 3×3×1 mm
Minimal cross-axis sensitivity between the accelerometer, gyroscope and magnetometer axes
512 byte FIFO buffer enables the application's processor to read the data in bursts
Digital-output temperature sensor
User-programmable digital filters for gyroscope, accelerometer, and temp sensor
10,000 g shock tolerant
400 kHz Fast Mode I2C for communicating with all registers
1 MHz SPI serial interface for communicating with all registers
20 MHz SPI serial interface for reading sensor and interrupt registers
MEMS structure hermetically sealed and bonded at wafer level
RoHS and Green compliant

Motion Processing
Internal Digital Motion Processing™ (DMP™) engine supports advanced Motion Processing and low power functions such as gesture recognition using programmable interrupts
Low-power movement pedometer functionality allows the host processor to sleep while the DMP maintains the
Movements count.

Element 19 of FIG. 4 is the device antenna. It uses a chip antenna or PCB antenna.

Element 20 of FIG. 4 is the device's memory flash. It uses for the device microcontroller and for storing all past data, as injection dosage, user smart phone Bluetooth smart address, Bluetooth smart wireless glucometer address settings etc.

Element 20A of FIG. 4 is the device's IR sensor; the TMP006 is digital temperature sensor that is optimal for thermal management and thermal protection applications where remote non-contact sensing is desired. Its package name is WCSP. The TMP006 are two-wire and SMBus interface compatible, and are specified over the ambient temperature range of −40° C. to +125° C. The TMP006 and TMP006B measure the user skin temperatures to detect penetration of the needle into the skin. The TMP006 contains registers for holding configuration information, temperature measurement results, and sensor voltage measurement. Ambient of the pen temperature and sensor battery voltage measurements are used to calculate the object temperature. The SCL and SDA interface pins require pull-up resistors (10 kΩ, typical) as part of the communication bus, while DRDY is an open-drain output that must also use a pull-up resistor. DRDY may be shared with other devices if desired for a wired-OR implementation. A 0.01-μF power-supply bypass capacitor is recommended, The TMP006 provides both local insulin pen body temperature and IR thermocouple sensor voltage outputs in a WCSP.

The local temperature sensor in TMP006 is integrated on-chip; the thermal path runs through the WCSP solder balls. The low thermal resistance of the solder balls provides the thermal path to maintain the chip at the temperature of the local insulin pen environment.

The top side of the WCSP must face the object that is being measured with an unobstructed view in order to accurately measure the temperature. Refer to the devices Assembly (FIG. 3—denoted 7)

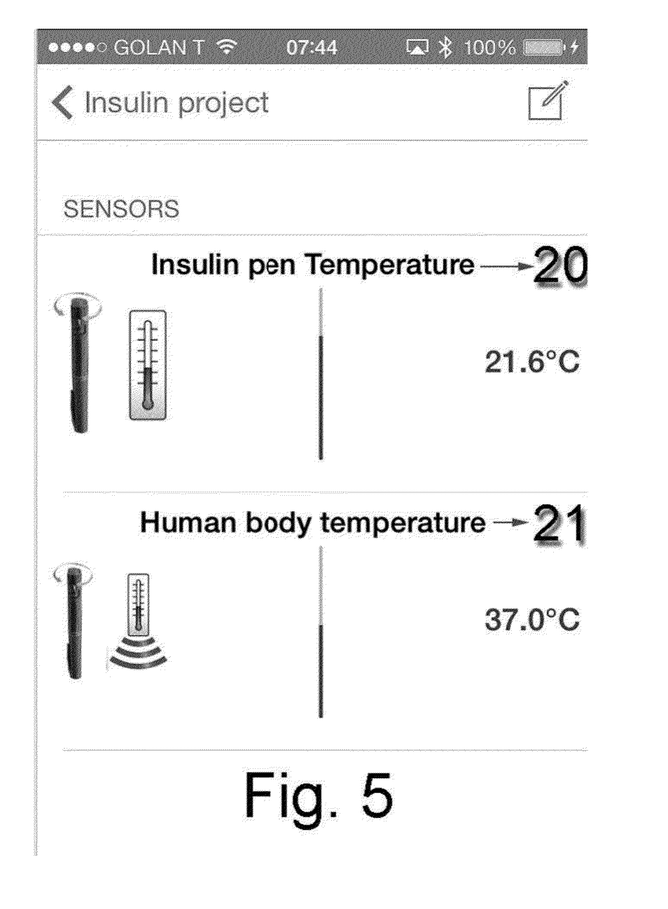

FIG. 5 illustrates a smart phone apps displays the device's insulin pen current self-temperature Element 20 of FIG. 5—If the past storage temperature was out of the manufacturer parameter the pen will alert the user and will display that bad storage temperature occurred. The insulin liquid is protein that destroyed by too high or too low temperatures. Injection insulin that changes can harm or kill the user. In this case, the device will sound and display error messages and will send an electronic alert to the user care taker. The user smart phone can sound an alert in this case with human voice.

The device's IR sensor (FIG. 4—denoted—denoted 20A) measurement is displayed on user's smart phone (FIG. 5—denoted 21). This sensor primary function is to detect penetration of the needle to the user skin, and stay inside 6 sec during injection.

Secondary function of the device's IR sensor (FIG. 4—denoted 20A) is its ability to deliver the user a smart phone screen (FIG. 4—denoted 20A) the skin temperature of organs that can suffer of low blood flowing, as often observed in this diabetes patient. Normally is observed on the legs. For example, if the user scans with the device his leg's fingers, and if detects a cool zone, he can rush to hospital before permanent necrosis occurs.

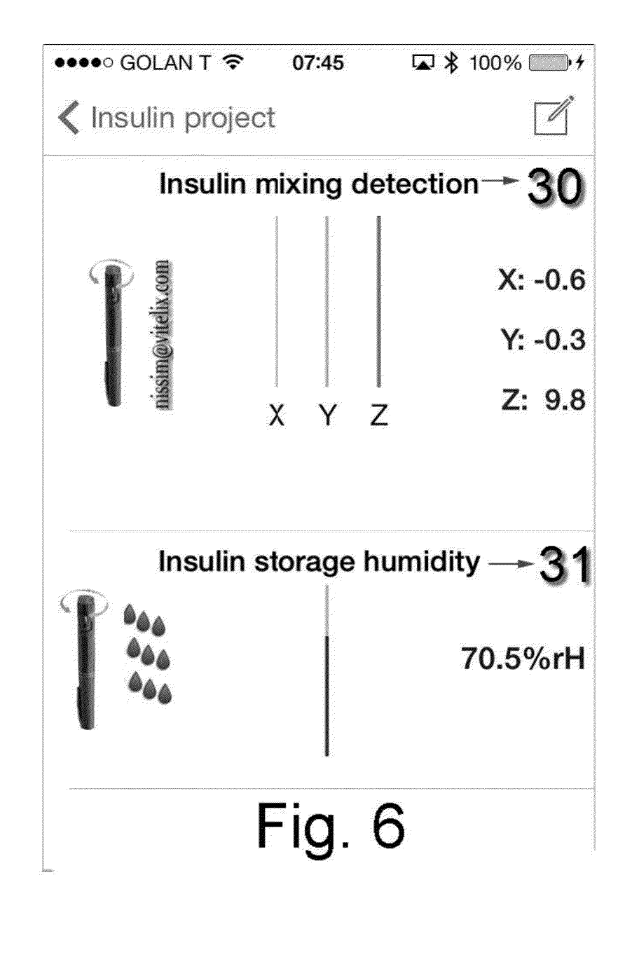

FIG. 6 illustrates a smart phone apps displays the device's insulin pen mixing (FIG. 6—denoted 30). by moving the pen up and down ten times. This information is revived from the device. The device detects it from sensor (FIG. 4—denoted 18).

The device has an option to detect the pen past storage humidity history (FIG. 6—denoted 31). If the past storage humidity was out of the manufacturer parameter the pen will alert the user and will display that bad storage humidity. The insulin liquid is protein that destroyed on too high or too low humidity. Injection insulin that changes can harm or kill the user. In this case, the device will sound and display error messages and will send an electronic alert to the user care taker.

The user smart phone can sound an alert in this case by human voice.

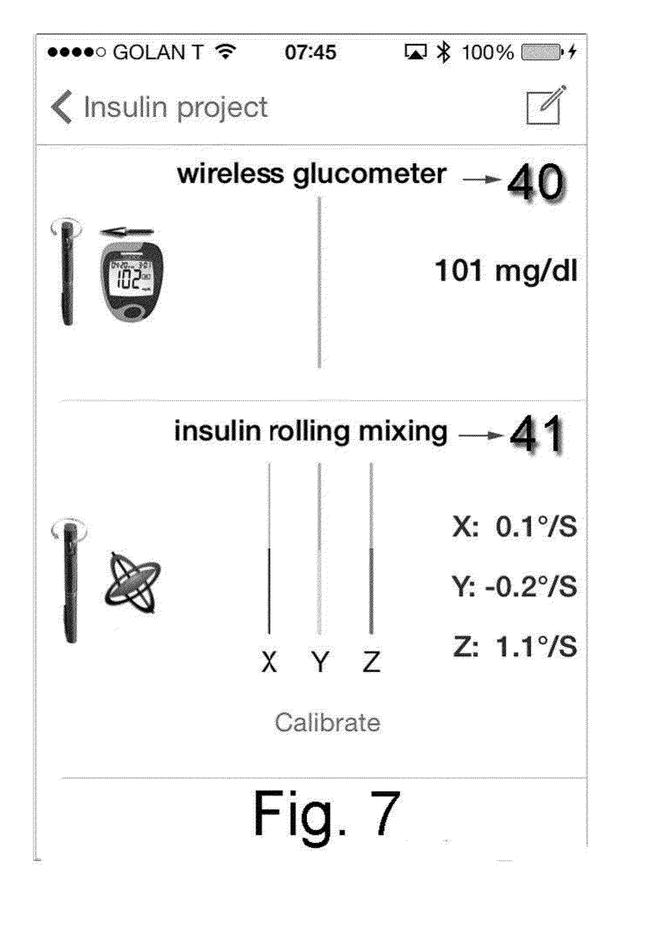

FIG. 7 illustrates a smart phone apps displays the device's last wirelessly read of blood glucose level (FIG. 7—denoted 40) of the user.

It also shows if the user properly rolls the pen between his palms 10 times (FIG. 7—denoted 41)—it is important that the pen is kept horizontally. This information is revived from the devices.

The device detects it from sensor (FIG. 4—denoted 18).

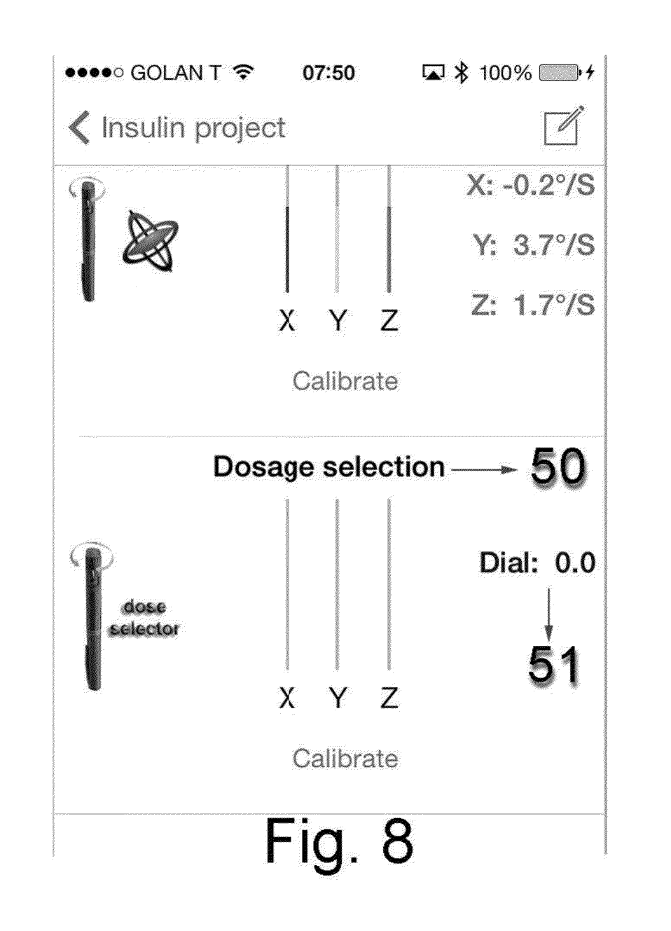

FIG. 8 illustrates a smart phone apps display the device's dosage selection after calibration (FIG. 8—denoted 50) before dial the value. It has to show “Dial:0.0” (FIG. 8—denoted 51).

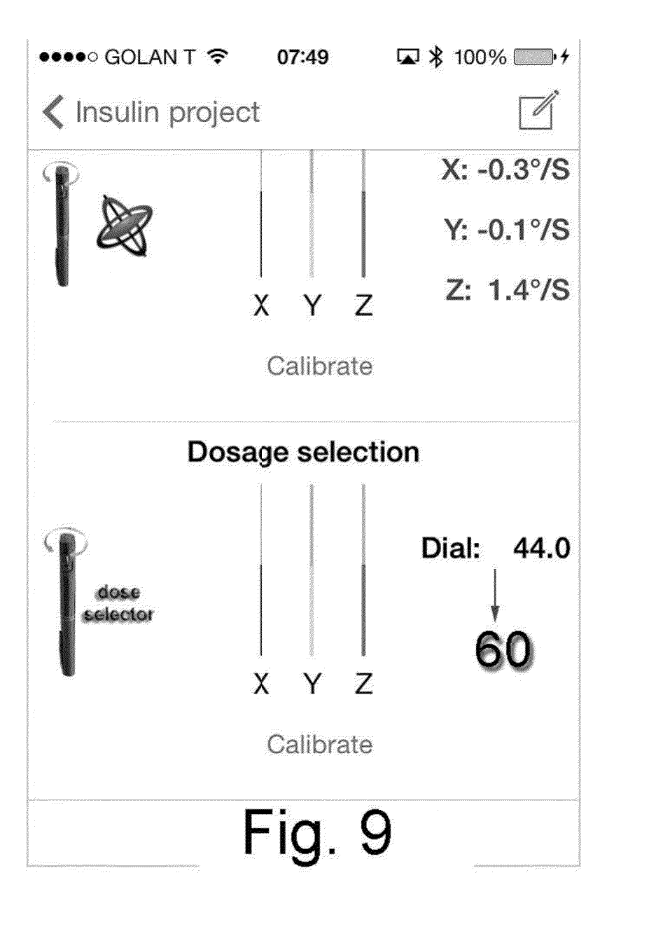

FIG. 9 illustrates a smart phone apps displays the device's dosage selection after user dial the value (FIG. 9—denoted 60). The user has to move the dial up, and when the dosage set to the right value the device sounds “beep” by its inner buzzer and/or plays a human voice message on user's smart phone speaker. When over dosage occurs, the device sounds alert buzzer sound and alert voice message on smart phone speaker. In this example the user has to dial 44. Led1 and Led2 (FIG. 4—denoted 13) will guide him to the right value without the need to open its smart phone.

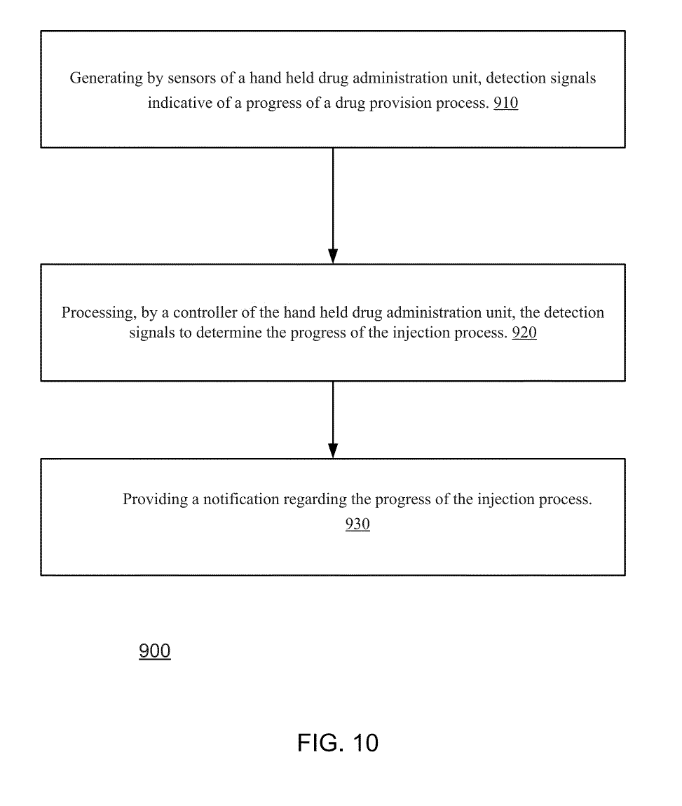

FIG. 10 illustrates a method 900 according to an embodiment of the invention.

Method 900 includes a sequence of stages 910, 920 and 930:
Stage 910 of generating by sensors of a hand held drug administration unit, detection signals indicative of a progress of a drug provision process.
Stage 920 of processing, by a controller of the hand held drug administration unit, the detection signals to determine the progress of the injection process.
Stage 930 of providing a notification regarding the progress of the injection process.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality. Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense. In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A hand held drug administration unit, comprising: a controller, sensors, a wireless transceiver, a memory unit, and a mechanical dosage and injection control unit that is controlled by a user;
wherein the sensors are configured to generate detection signals indicative of a progress of a drug provision process;
wherein the controller is configured to process the detection signals and to determine the progress of the injection process and to provide a notification regarding the progress of the injection process.

2. The hand held drug administration unit wherein the controller, the sensors and the wireless transceiver are either integrated within a housing of the hand held drug administration unit or are connected to the housing of the hand held drug administration unit.

3. The hand held drug administration unit according to claim 1 wherein the hand held drug administration unit is a drug injection pen.

4. The hand held drug administration unit according to claim 1 wherein the sensors comprise an orientation sensor configured to monitor an orientation of the hand held drug administration unit.

5. The hand held drug administration unit according to claim 4 wherein the controller is configured to determine, based upon detection signals from the orientations sensor, whether the user performs predetermined movements that are mandatory to the drug provision process.

6. The hand held drug administration unit according to claim 4 wherein the orientation sensor is a three axis accelerometer.

7. The hand held drug administration unit according to claim 1 wherein the sensors comprise at least one mechanical dosage and control unit sensor that is configured to sense a movement of the mechanical dosage and control unit.

8. The hand held drug administration unit according to claim 7 wherein the controller is configured to determine, based upon, at least, detection signals from the at least one mechanical dosage and control unit sensor, a target amount of dosage to be administered during the drug provision process and an actual amount of drug that is administered by the user.

9. The hand held drug administration unit according to claim 7 wherein the at least one mechanical dosage and control unit sensor comprises a magnetic element connected to the mechanical dosage and control unit and a static magnetic flux reader.

10. The hand held drug administration unit according to claim 7 wherein the sensors comprise a temperature sensor for sensing when the drug is being injected, wherein the controller is configured to determine, based upon, at least, detection signals from the temperature sensor and at the least one mechanical dosage and control unit sensor, a target amount of dosage to be administered during the drug provision process and an actual amount of drug that is administered by the user.

11. The hand held drug administration unit according to claim 1 wherein the sensor comprise a temperature sensor for sensing when the drug is being injected.

12. The hand held drug administration unit according to claim 1 wherein the memory unit stores a set of instructions to be followed by the user when administering the drug, wherein the controller is configured to determine whether the user follows the instructions and send an alert when the user deviates from the instructions.

13. The hand held drug administration unit according to claim 1 wherein the wireless transceiver is configured to communicate with a user device that has a man machine interface for providing to the user indications about the progress of the drug administration process.

14. The hand held drug administration unit according to claim 1 wherein the controller is configured to receive information from a user monitor about a status of the user and to send an alert if the user did not administer the drug in accordance to the status of the user.

15. The hand held drug administration unit according to claim 13 wherein the controller is configured to generate an alert is the user did not administer a drug when the status of the user requires an administration of the drug.

16. The hand held drug administration unit according to claim 13 wherein the controller is configured to administer an alert when the user sets the mechanical dosage and injection control unit to administer an amount of drug that is too high to the status of the user.

17. A method for administering drug, the method comprises: generating by sensors of a hand held drug administration unit, detection signals indicative of a progress of a drug provision process; processing, by a controller of the hand held drug administration unit, the detection signals to determine the progress of the injection process and, providing a notification regarding the progress of the injection process.

18. A kit comprising a controller, sensors, a wireless transceiver, a memory unit; wherein when at least one of the sensors are coupled to a mechanical dosage and injection control unit that is controlled by a user and belongs to a hand held drug administration unit, the at least one of the sensors is configured to generate detection signals indicative of a progress of a drug provision process; and the controller is configured to process the detection signals and to determine the progress of the injection process and to provide a notification regarding the progress of the injection process.
